(19) United States
(12) Patent Application Publication
(10) Pub. No.: US 2024/0398212 A1
(43) Pub. Date: Dec. 5, 2024

(54) SURGICAL SCOPE ADAPTER FOR ACTUATED MANEUVERING OF SCOPES

(71) Applicants: Qatar Foundation for Education, Science and Community Development, Doha (QA); Hamad Medical Corporation, Doha (QA)

(72) Inventors: Nikhil Vishwas Navkar, Doha (QA); Carlos Velasquez, Doha (QA); Diya Abdelmagid, Doha (QA); Jhasketan Padhan, Doha (QA); Shidin Balakrishnan, Doha (QA); Julien Abi Nahed, Doha (QA); Mohammed Khorasani, Doha (QA); Abdulla Al Ansari, Doha (QA)

(21) Appl. No.: 18/699,815

(22) PCT Filed: Oct. 11, 2022
(86) PCT No.: PCT/QA2022/050021

§ 371 Date: Apr. 9, 2024

Related U.S. Application Data
(60) Provisional application No. 63/254,351, filed on Oct. 11, 2021.

(51) Int. Cl. A61B 1/00; A61B 34/30

(52) U.S. Cl. CPC A61B 1/00149; A61B 1/0014; A61B 1/0016; A61B 34/30

(57) ABSTRACT

The present disclosure relates to a scope adapter for adapting a surgical scope to a robotic manipulator system. In particular, the disclosure relates to system and methods of using the scope adapter including a rotational motor mounted in a fixed position relative to an outer cylinder, an inner cylinder rotatably coupled to the outer cylinder, and a support plate assembly that is removably coupled to the surgical scope and the inner cylinder.

### CROSS-REFERENCE TO RELATED APPLICATIONS

This application claims priority to and the benefit of U.S. Provisional Patent Application No. 63/254,351 filed Oct. 11, 2021, the entire contents of which are incorporated by reference herein.

### BACKGROUND

During a minimally invasive surgery (“MIS”), a scope with an attached camera can be used to see inside of the patient's body. To focus the camera's field-of-view (“FOV”), the scopes can be maneuvered, which provides greater flexibility to operate on hard-to-reach anatomical regions. These scopes are shown in FIGS. 1-3 and can be broadly categorized into three categories based on visualization and maneuvering capabilities. In particular, FIG. 1 illustrates a zero-degree scope 10, FIG. 2 illustrates an angulated scope 20, and FIG. 3 illustrates an articulated scope 30 (each alternatively referred to generally as “scopes”). In each example, how the scope 10, 20, 30 is maneuvered influences the FOV provided to the user.

To insert one of the scopes 10, 20, 30 into a patient 1, an incision 2 is made in the patient 1 (typically by a cutting trocar not shown) and a cannula 4 is placed in the incision 2 to provide a pathway for the scopes 10, 20, 30 insertion. In the example of FIG. 1, a first end 10a of the scope 10 is pushed into the patient by manipulating a second end 10b. In particular, the scope 10 is moved into and out of the cannula 4 via an insertion or retraction 6, is angulated back and forth in a first plane by a tilt up 8 and a tilt down 12, and is angulated back and forth in a second plane by a pan left 14 and a pan right 16. Using combinations of manipulations applied at the second end 10b, the user of the scope 10 can change the position of the FOV 19, which is fixed relative to an axis 15 for the zero-degree scope 10. The scope 10 may also be rotated about the axis 15, but the extent of the FOV 19 is unchanged during rotation when the FOV 19 is symmetric relative to the axis 15.

Referring to FIG. 2, a first end 20a of the angulated scope 20 may also be manipulated via a second end 20b in the same manner described for the scope 10. However, a rotation 28 imparted to the angulated scope 20 about the axis 15 does vary a direction 25 of a FOV 29 because the FOV 29 is oriented at a fixed angle relative to the axis 15. Angulated scopes 20 are available in different fixed angles as shown by an angle 27 between the direction 25 and the axis 15.

Referring to FIG. 3, a first end 30a of the articulated scope 30 may also be manipulated via a second end 30b in the same manner described for the scopes 10, 20. The articulated scope 30 is similar to the scope 20 in that a direction 35 of a FOV 39 may be adjusted by imparting a rotation 38 to the second end 30b. However, the articulated scope 30 differs from the scope 20 in that the direction 35 may also be adjusted by varying an adjustable angle 37. As shown by FIG. 4, the selection of the scope 10, 20, 30 may be based on the specific FOV 19, 29, 39 needed to operate in a targeted tissue region 40.

To enhance the effectiveness of using the scopes 10, 20, 30, a robotic manipulator system may also be used to provide a steady hold and to maneuver the scopes 10, 20, 30 by the second ends 10b, 20b, 30b. In particular, the robotic manipulator system may be used for insertion and retraction 6, tilt up 8, tilt down 12, pan left 14, and pan right 16. However, the ability for the robotic manipulator system to impart the rotations 28, 38 and to vary the adjustable angle 37 are limited due to a lack of compatibility of the scope holder that is used to interface between the robotic arm and the scopes 10, 20, 30. In addition, the scope holders that are available are each only compatible with one type of scope and are thus limited in their ability to switch between the scopes 10, 20, 30. Therefore, the development of scope adapters that facilitate usage of all scopes types with a robotic manipulator system is needed.

### SUMMARY

The present disclosure generally relates to a surgical scope adapter for actuated maneuvering of scopes.

In light of the present disclosure, and without limiting the scope of the disclosure in any way, in a first aspect of the present disclosure, which may be combined with any other aspect listed herein unless specified otherwise, a scope adapter for adapting a surgical scope to a robotic manipulator system, the scope adapter comprising a rotational motor mounted in a fixed position relative to an outer cylinder, an inner cylinder rotatably coupled to the outer cylinder, and a support plate assembly that is removably coupled to the surgical scope and the inner cylinder.

In accordance with a second aspect of the present disclosure, which may be used in combination with any other aspect listed herein unless stated otherwise, the scope adapter, wherein the inner cylinder is rotatable concentrically inside the outer cylinder.

In accordance with a third aspect of the present disclosure, which may be used in combination with any other aspect listed herein unless stated otherwise, the scope adapter further comprising a belt operatively coupled to the rotational motor, wherein the belt circumferentially wraps around a portion of an outer diameter of the inner cylinder, and wherein the belt is configured to transfer rotation from the rotational motor to the inner cylinder.

In accordance with a forth aspect of the present disclosure, which may be used in combination with any other aspect listed herein unless stated otherwise, the scope adapter further comprising a shelf extending from an inner diameter of the inner cylinder, where the shelf comprises a groove that is slidably couplable with a ridge extending from the support plate.

In accordance with a fifth aspect of the present disclosure, which may be used in combination with any other aspect listed herein unless stated otherwise, the scope adapter further comprising a plurality of spherical bearings located between the inner cylinder and the outer cylinder.

In accordance with a sixth aspect of the present disclosure, which may be used in combination with any other aspect listed herein unless stated otherwise, the scope adapter further comprising a scope gear fixably coupled to the surgical scope, and an articulation motor rotatably coupled to an articulation gear, wherein the articulation gear is operably coupled to the scope gear and is configured to transfer rotations of the articulation motor to the scope gear, and thereby change an adjustable direction of a field of view for the surgical scope.

In accordance with a seventh aspect of the present disclosure, which may be used in combination with any other aspect listed herein unless stated otherwise, the scope adapter wherein the support plate assembly further comprises a front base with a front aperture passing therethrough along a central axis of the inner cylinder, and a rear base with a rear aperture passing therethrough, wherein the front aperture and the rear aperture are configured to fixably couple to the surgical scope.

In accordance with a eighth aspect of the present disclosure, which may be used in combination with any other aspect listed herein unless stated otherwise, the scope adapter wherein the front aperture is at least partially cylindrical, and wherein the rear aperture is positioned along an axis that is non-parallel with respect to the central axis of the inner cylinder.

In accordance with a ninth aspect of the present disclosure, which may be used in combination with any other aspect listed herein unless stated otherwise, the scope adapter wherein the front base rotatably couples to a front cap with a front hinge, the rear base rotatably couples to a rear cap with a rear hinge, and wherein the front aperture opens by operation of the front hinge, and the rear aperture opens by operation of the rear hinge.

In accordance with a tenth aspect of the present disclosure, which may be used in combination with any other aspect listed herein unless stated otherwise, a method of adapting a surgical scope to a robotic manipulator system with a scope adapter, the method comprising coupling the surgical scope to a support plate assembly, coupling the support plate assembly to an inner cylinder, operating a rotational motor to rotate the inner cylinder concentrically within an outer cylinder, and rotating a portion of the surgical scope relative to the outer cylinder.

In accordance with an eleventh aspect of the present disclosure, which may be used in combination with any other aspect listed herein unless stated otherwise, the method further comprising rotating a belt circumferentially around an outer diameter of the inner cylinder to transfer rotation from the rotational motor to the inner cylinder.

In accordance with a twelfth aspect of the present disclosure, which may be used in combination with any other aspect listed herein unless stated otherwise, the method further comprising fixably coupling a scope gear to the surgical scope, and operating an articulation motor coupled to an articulation gear to transfer rotations of the articulation motor to the scope gear, and thereby changing an adjustable direction of a field of view for the surgical scope.

In accordance with a thirteenth aspect of the present disclosure, which may be used in combination with any other aspect listed herein unless stated otherwise, the method further comprising coupling the surgical scope to a front base with a front aperture passing therethrough along a central axis of the inner cylinder, and coupling the surgical scope to a rear base with a rear aperture passing therethrough.

In accordance with a fourteenth aspect of the present disclosure, which may be used in combination with any other aspect listed herein unless stated otherwise, the method further comprising opening the front aperture by rotating a front cap with a front hinge on the front base and opening the rear aperture by rotating a rear cap with a rear hinge on the rear base.

In accordance with a fifteenth aspect of the present disclosure, which may be used in combination with any other aspect listed herein unless stated otherwise, a scope adapter for adapting a surgical scope to a robotic manipulator system, the scope adapter comprising a rotational motor mounted in a fixed position relative to an outer cylinder, an inner cylinder rotatably coupled to the outer cylinder, a rear inner cylinder concentrically aligned with the inner cylinder; a front support plate that is removably coupled to the surgical scope and the inner cylinder, and a rear support plate that is removably coupled to the surgical scope and the rear inner cylinder.

In accordance with a sixteenth aspect of the present disclosure, which may be used in combination with any other aspect listed herein unless stated otherwise, the scope adapter wherein the inner cylinder is rotatable concentrically inside the outer cylinder and relative to a rational position of the rear inner cylinder.

In accordance with a seventeenth aspect of the present disclosure, which may be used in combination with any other aspect listed herein unless stated otherwise, the scope adapter further comprising a belt operatively coupled to the rotational motor, wherein the belt circumferentially wraps around a portion of an outer diameter of the inner cylinder, and wherein the belt is configured to transfer rotation from the rotational motor to the inner cylinder.

In accordance with an eighteenth aspect of the present disclosure, which may be used in combination with any other aspect listed herein unless stated otherwise, the scope adapter further comprising a shelf extending from a first inner diameter of the inner cylinder, where the shelf comprises a groove that is slidably couplable with the front support plate, and a rear shelf extending from a second inner diameter of the rear inner cylinder, where the rear shelf comprises a rear groove that is slidably couplable with the rear support plate.

In accordance with a nineteenth aspect of the present disclosure, which may be used in combination with any other aspect listed herein unless stated otherwise, the scope adapter wherein the front support plate further comprises a front base with a front aperture passing therethrough along a central axis of the inner cylinder, and the rear support plate further comprises a rear base with a rear aperture passing therethrough, wherein the front aperture and the rear aperture are configured to fixably couple to the surgical scope.

In accordance with a twentieth aspect of the present disclosure, which may be used in combination with any other aspect listed herein unless stated otherwise, the scope adapter wherein the rear aperture is positioned along an axis that is parallel with respect to the central axis of the inner cylinder.

The reader will appreciate the foregoing details, as well as others, upon considering the following detailed description of certain non-limiting embodiments including an apparatus for adapting surgical scopes.

### BRIEF DESCRIPTION OF THE DRAWINGS

These and other features, aspects, and advantages of certain embodiments will become better understood when the following detailed description is read with reference to the accompanying drawings in which like characters represent like parts throughout the drawings, wherein:

FIG. 1 is a schematic view of a prior art zero degree scope used to perform minimally invasive surgery;

FIG. 2 is a schematic view of a prior art angulated scope used to perform minimally invasive surgery;

FIG. 3 is a schematic view of a prior art articulated scope used to perform minimally invasive surgery;

FIG. 4 is a schematic view of an example use for the prior art scopes of FIGS. 1-3;

FIG. 5 is a perspective view of a robotic manipulator system including a scope adapter, according to one or more embodiments;

FIG. 6 is a schematic front view of a scope adapter, according to one or more embodiments;

FIG. 7 is a schematic side view of a scope adapter, according to one or more embodiments;

FIG. 8 is a perspective view of a support plate assembly that may be used with a scope adapter, according to one or more embodiments;

FIG. 9 is another perspective view of the support plate assembly of FIG. 8;

FIG. 10 is a perspective view of the support plate assembly of FIG. 8 coupled with a scope;

FIG. 11 is another perspective view of the support plate assembly and the scope of FIG. 10;

FIG. 12 is a perspective view of the support plate assembly of FIG. 8 coupled with the scope and the scope adapter;

FIG. 13 is another perspective view of the support plate assembly coupled with the scope and the scope adapter of FIG. 12;

FIG. 14 is a perspective view of another support plate assembly, according to one or more embodiments;

FIG. 15 is another perspective view of the support plate assembly of FIG. 14;

FIG. 16 is a perspective view of the support plate assembly of FIG. 14 coupled with a scope;

FIG. 17 is another perspective view of the support plate assembly and the scope of FIG. 16; and

FIG. 18 is a schematic side view of another scope adapter, according to one or more embodiments.

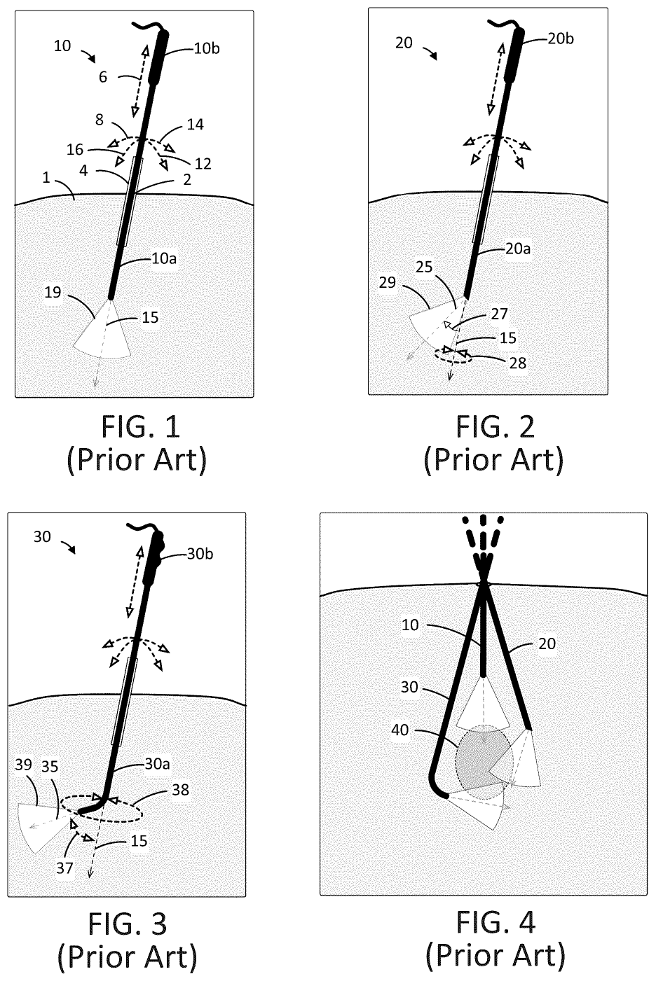

### DETAILED DESCRIPTION

The present disclosure generally relates to an apparatus for adapting surgical scopes to robotic manipulator systems.

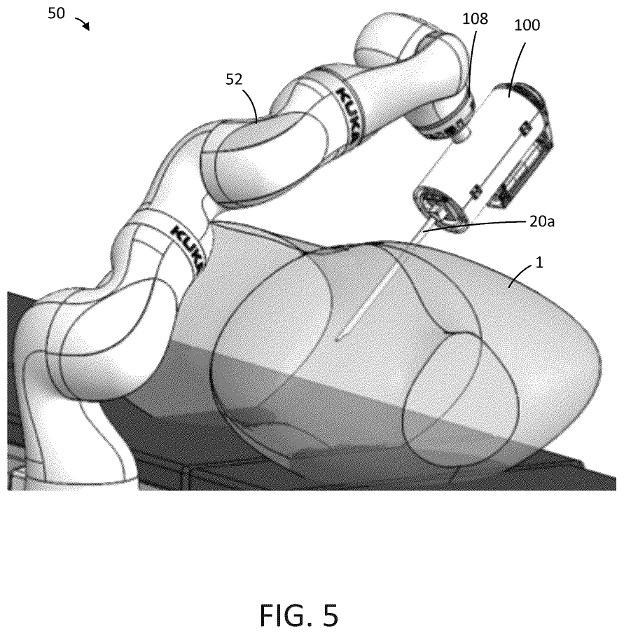

Referring to FIG. 5, a robotic manipulator system 50 including a robotic arm 52 is shown equipped with a scope adapter 100. As described herein the scope adapter 100 is configurable to interchangeably adapt to the zero degree scope 10, the angulated scope 20, and the articulated scope 30. In addition, the scope adapter 100 may be configured to impart the rotation 28 for the angulated scope 20 (as shown in FIG. 2), to selectably adjust the rotation 38, and to selectably adjust the adjustable angle 37 for the articulated scope 30 (as shown in FIG. 3).

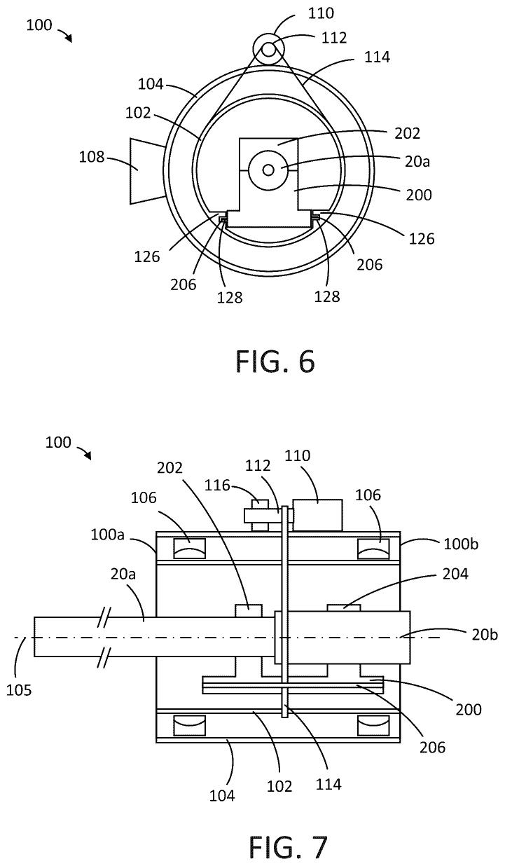

Referring to FIGS. 6 and 7, a schematic front view and a schematic side view, respectively, of the scope adapter 100 are shown with some details simplified or exaggerated in size to illustrate the general configuration. As shown, the scope adapter 100 comprises an inner cylinder 102 within and concentrically aligned with an outer cylinder 104. The inner cylinder 102 is supported within the outer cylinder 104 by a pair of spherical bearings 106 such that the inner cylinder 102 is able to rotate around a central axis 105 while the outer cylinder 104 is fixed and is connected to the robotic arm 52 via the base 108. The spherical bearings 106 may be held in position by a press fit, bonding, a series of stepped diameters within one or both of the cylinders 102, 104 and may be used in combination with retention rings (e.g., “snap rings”) not shown.

A rotational motor 110 mounts in a fixed position on the outer cylinder 104. The rotational motor 110 may include a reduction gearbox (not shown) that may be used to increase or decrease the rotational speed of a shaft 112 extending therefrom. Optionally, an encoder (not shown) coupled to the rotational motor 110 or the shaft 112 may also be used to provide angular speed and/or angular position feedback. A belt 114 is operatively coupled around the shaft 112 and is operatively coupled to an outer diameter of the inner cylinder 102 by circumferentially wrapping around a portion of the inner cylinder 102. A thru hole (not shown) in the wall of the outer cylinder 104 allows passage of the belt 114. The belt 114 may be smooth, textured, or contain teeth, ribs, cogs, or other “features” along the length of the belt 114 to provide positive engagement between the belt 114, the shaft 112, and the outer diameter of the inner cylinder 102. To accommodate the features of the belt 114, the shaft 112 may also include mating features such as a gear or splines (not shown) and the outer diameter of the inner cylinder 102 may also include similar mating features. An optional support block 116 including bushings or bearings may also be rotatably coupled to the shaft 112 to provide bending support to the shaft 112 as the belt 114 is tensioned and operates. While not shown in the example of FIGS. 6 and 7, it is also anticipated that no belt 114 may be used and instead gears mounted on the shaft 112 may directly interface with gears mounted on the outer diameter of the inner cylinder 102. Therefore, either with or without the belt 114, the operation of the rotational motor 110 is configured to apply torque to and rotate the inner cylinder 102 relative to the outer cylinder 104.

Referring still to FIGS. 6 and 7, the scope adapter 100 further comprises a support plate assembly 200 that is removably coupled to the inner cylinder 102 and is removably coupled to any of the surgical scopes 10, 20, 30, previously described. The geometry and configuration of the support plate assembly 200 can be adjusted to accommodate any of the scopes 10, 20, 30 and the coupling to the simplified angulated scope 20 in FIGS. 6 and 7 is exemplary only. As shown in FIGS. 6 and 7, the support plate assembly 200 comprises a front cap 202 to couple with the first end 20a of the scope 20 and comprises a rear cap 204 to couple with the second end 20b of the scope 20. As best shown in FIG. 6 for the front cap 202, the caps 202, 204 may bolt down or otherwise attach to the support plate assembly 200 and thereby capture and attach the scope 20 to the support plate assembly 200. As best shown in the side view of FIG. 7, the sizes of the caps 202, 204 and the connecting portions of the support plate assembly 200 may be adjusted as needed so that the central axis of the first end 20a coincides with the central axis 105 of the scope adapter 100.

To accurately locate the position of the support plate assembly 200 (including accurately establishing the concentric alignment of the central axis of the first end 20a with the central axis 105), slidably mating features between the inner cylinder 102 and the support plate assembly 200 may be used. In this manner, the support plate assembly 200 may be repeatedly installed and removed (e.g., coupled and decoupled) from connection with the inner cylinder 102 while returning to a substantially similar position on each installation. More specifically, the inner cylinder 102 may comprise two or more shelves 126, each comprising a groove 128 extending into each shelf 126 and extending substantially parallel with the central axis 105. Additionally, the support plate assembly may further comprise two or more ridges 206 that also extend substantially parallel with the central axis 105 when installed therewith. When connecting the support plate assembly 200 with the inner cylinder 102, the ridges 206 slidably couple with the grooves 128 and thus guide the position of the support plate assembly 200.

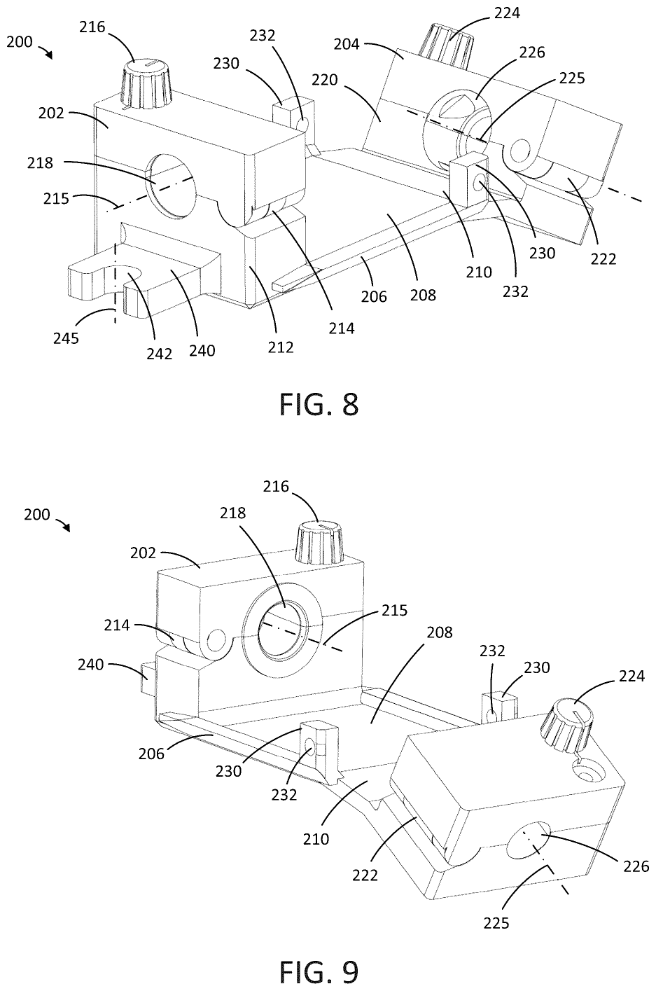

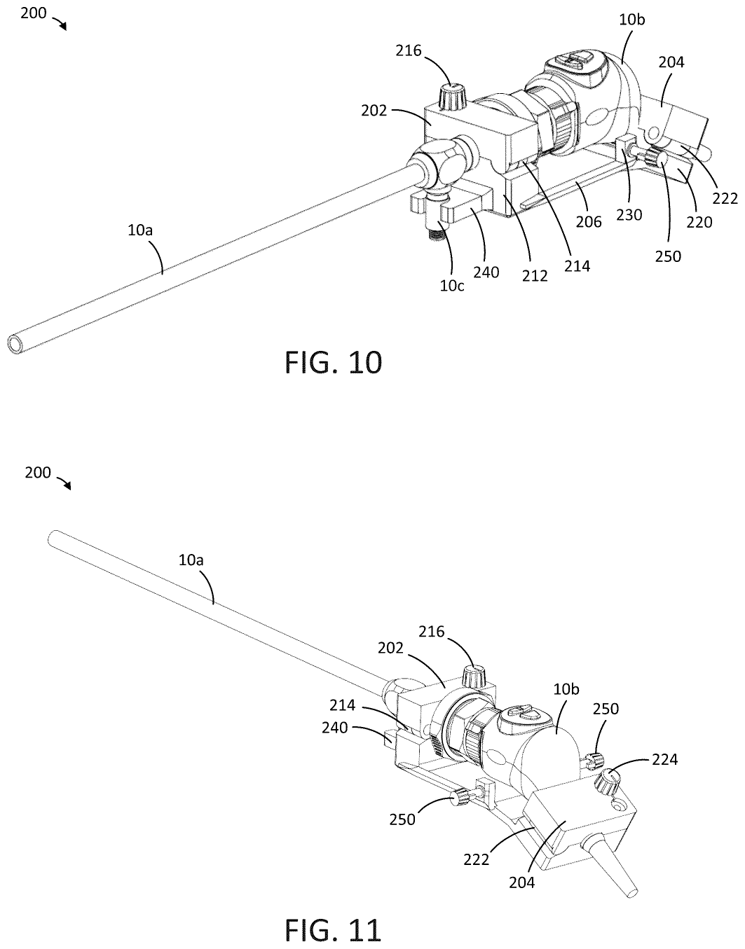

Referring to FIGS. 8 and 9, two isometric views of the support plate assembly 200 are shown in greater detail and include a “bend” along a lower portion to accommodate a scope 10, 20, 30 that is formed with a similar bent configuration (as shown in the examples of FIGS. 10 and 11). The particular the support plate assembly 200 comprises a front plate 208 which is substantially parallel with a central axis 215 and a rear plate 210 that forms a non-zero angle with respect to the central axis 215. The central axis 215 is concentric with the central axis 105 of the inner cylinder 102 when installed therewith, and thus the rear plate 210 is non-parallel with respect to the central axis 105. The angle between the front plate 208 and the rear plate 210 may be adjusted as needed to adjust to the geometry of commercially available scopes 10, 20, 30. To provide a platform to support the scopes 10, 20, 30, a front base 212 extends from the front plate 208 and includes a front hinge 214 that rotatably connects with the front cap 202. A front knob 216 restricts the front hinge 214 motion and maintains the front cap 202 position relative to the front base 212. A front aperture 218 extends through both the front cap 202 and the front base 212 in a direction substantially parallel with the central axis 215. In the example of FIGS. 8 and 9, the front aperture 218 is at least partially cylindrical and is concentrically positioned with the central axis 215. The front aperture 218 may also include a series of steps, tapering surfaces, or recesses as needed to adapt to the particular scope 10, 20, 30.

To provide another platform to support the scopes 10, 20, 30, a rear base 220 extends from the rear plate 210 and includes a rear hinge 222 that rotatably connects with the rear cap 204. A rear knob 224 restricts the rear hinge 222 motion and maintains the rear cap 204 position relative to the rear base 220. A rear aperture 226 extends through both the rear cap 204 and the rear base 220 in a direction substantially parallel with the rear plate 210 as shown by an axis 225. In the example of FIGS. 8 and 9, the rear aperture 226 is at least partially cylindrical and is concentrically positioned with the axis 225. As best shown in FIG. 8, the rear aperture 226 also includes a series of steps and recesses within the cylindrical walls forming the rear aperture 226. Similar to the front aperture 218 previously described, the rear aperture 226 may include any combination or series of steps, tapering surfaces, or recesses as needed to adapt to the particular scope 10, 20, 30.

It is anticipated that the sizes of the front aperture 218 and the rear aperture 226 may be adjusted as needed to provide a firm and substantially fixed position relative to the scopes 10, 20 30. In addition, the angle between the central axis 215 and the axis 225 provide a certain degree of mechanical capture that will restrict sliding motion along axes 215, 225. However, the scopes 10, 20, 30 may also be attached to the support plate assembly 200 at additional points. Further, it is anticipated that additional points may be used to angularly orient the scopes relative to the central axis 215 and may be used to establish a consistent translation position as the support plate assembly 200 is slid into position within the grooves 128 of the scope adapter 100. Such additional points of connection on the support plate assembly 200 comprise tabs 230 having pinholes 232 therethrough and a post arm 240 having a post aperture 242 therethrough along an axis 245.

Referring to FIGS. 10 and 11, the support plate assembly 200 is again shown in two different isometric views and includes the zero degree scope 10 installed therewith. As shown, the support plate assembly 200 is configurable to fixably couple to the scope 10 by using the front base 212 to clamp the first end 10a and by using the rear base 220 to clamp to the second end 10b. In addition, a light post 10c of the scope 10 is also captured by the post aperture 242 of the post arm 240 and thus further aligns the angular position of the scope 10 relative to the central axis 215. As shown in FIGS. 10 and 11, a locking pin 250 is shown installed with the pinholes 232 of the tabs 230 via threads within the pinholes 232. Optionally, the pinholes 232 may not be threaded or may not fully extend through the tabs 230 (e.g., blind holes) or may be spherical detents.

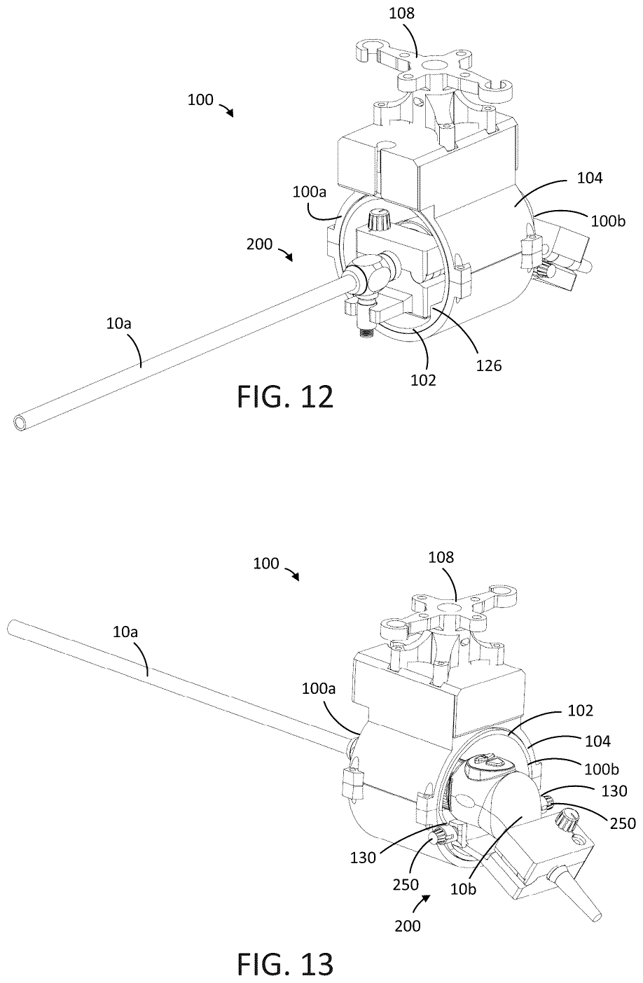

Referring to FIGS. 12 and 13, the support plate assembly 200 is again shown in two different isometric views and includes the zero degree scope 10 installed therewith. In addition, the support plate assembly 200 is shown coupled to the inner cylinder 102 of the scope adapter 100. After connecting the scope 10 with the support plate assembly 200 as shown in FIGS. 10 and 11, the scope 10 and the support plate assembly 200 are slidably coupled to the inner cylinder 102 via access along the second end 100b of the scope adapter 100. The cables extending from the scope 10 (e.g., video cables, power cables, etc.) may already be connected to the surgical video monitors (not shown) when the support plate assembly 200 is installed via the second end 100b. As best shown in FIG. 13, as the support plate assembly 200 is slid into the inner cylinder 102, and the extent of the sliding translation is limited by the locking pins 250 contacting extensions 130 that extend from the inner cylinder 102. When the locking pins 250 are coupled to the tabs 230 (e.g., threadably fixed to the tabs 230), the extensions 130 may also include latches that then capture the locking pins 250. In this manner, the slidable position between the support plate assembly 200 and the inner cylinder 102 are fixed. Alternatively, the locking pins 250 may instead be coupled to the extensions 130 of the inner cylinder 102 and the ends of the locking pins 250 may extend inward to interface with the pinholes 232. For example the locking pins 250 may be spring loaded detents, where the hemi-spherical head of the locking pins 250 locks into a fixed position within the pinholes 232. Alternatively, the locking pins 250 may be threaded at the coupling with the extensions 130 and may be threadably advanced into engagement with the pinholes 232.

Still further, the support plate assembly 200 may alternatively be installed via the first end 100a of the scope adapter 100. In this instance, the cables extending from the scope 10 (e.g., video cables, power cables, etc.) may be passed from the first end 100a to the second end 100b, and the support plate assembly 200 would then be slid into engagement with the inner cylinder 102 until the tabs 230 came into alignment with the locking pins 250 and the extensions 130. After installing the support plate assembly 200, the cables extending from the scope 10 could then be connected to the surgical video monitors (not shown).

By any of the described methods, the support plate assembly 200 may be removably coupled to the scopes 10, 20, 30 and may be removably coupled to the inner cylinder 102. Thus by the operation of the rotational motor 110, the scopes 10, 20, 30 may be rotated with the inner cylinder 102 relative to the outer cylinder 104 to adjust the FOV 19, 29, 39 as needed for a particular surgical procedure.

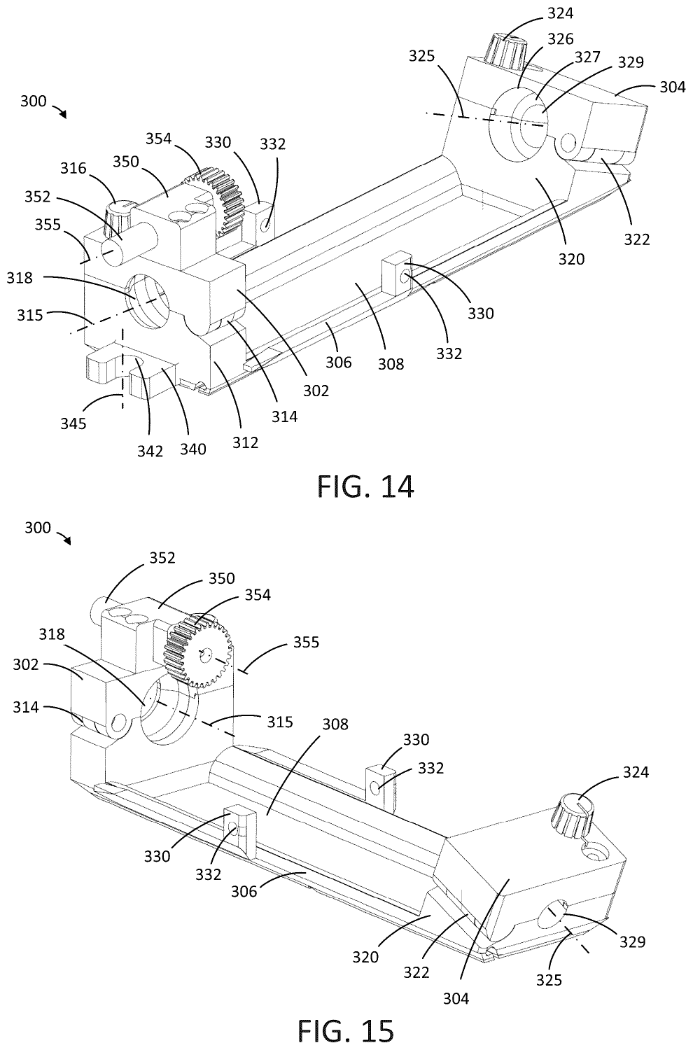

Referring to FIGS. 14 and 15, a support plate assembly 300 is shown in two different isometric views. The support plate assembly 300 is similar to the support plate assembly 200 previously described and may be used in place of the support plate assembly 200 to adapt to other types of scopes such as the articulated scope 30. As such, similar reference numerals are used to identify similar features and the explanation of such features will not be repeated in the interest of brevity except as needed for clarity. Instead, the description will focus primarily on aspects of the support plate assembly 300 that are different from the support plate assembly 200. Generally speaking, the support plate assembly 300 includes a base that is planar rather than being formed along two planes as described for the support plate assembly 200. In addition, the support plate assembly 300 includes components that will allow selectable adjustments to the adjustable angle 37 for the articulated scope 30 (as shown in FIG. 3).

In particular, the support plate assembly 300 comprises a front plate 308 which is substantially parallel with a central axis 315 of a front aperture 318. The central axis 315 is concentric with the central axis 105 of the inner cylinder 102 when installed therewith. To provide a platform to support the scopes 10, 20, 30, a front base 312 extends from the front plate 308 and includes a front hinge 314 that rotatably connects with a front cap 302. A front knob 316 restricts the front hinge 314 motion and maintains the front cap 302 position relative to the front base 312. A front aperture 318 extends concentrically with the central axis 315 through both the front cap 302 and the front base 312. The front aperture 318 may also include a series of steps, tapering surfaces, or recesses as needed to adapt to the particular scope 10, 20, 30.

To provide another platform to support the scopes 10, 20, 30, a rear base 320 extends from the front plate 308 at a position spaced apart from the front base 312. The rear base 320 may extend orthogonally relative to the central axis 315 or may extent at an angle relative to the central axis 315 as shown in the example of FIGS. 14 and 15. A rear hinge 322 rotatably connects the rear base 320 and the rear cap 304. A rear knob 324 restricts the rear hinge 322 motion and maintains the rear cap 304 position relative to the rear base 320. A rear aperture 326 extends through both the rear cap 304 and the rear base 320 along an axis 325 and at the angle needed to adapt to the scope 10, 20, 30 geometry. As best shown in FIG. 14, the rear aperture 326 is at least partially cylindrical and includes a series of steps. Alternatively, the rear aperture 326 may include any combination or series of steps, tapering surfaces, or recesses as needed to adapt to the particular scope 10, 20, 30.

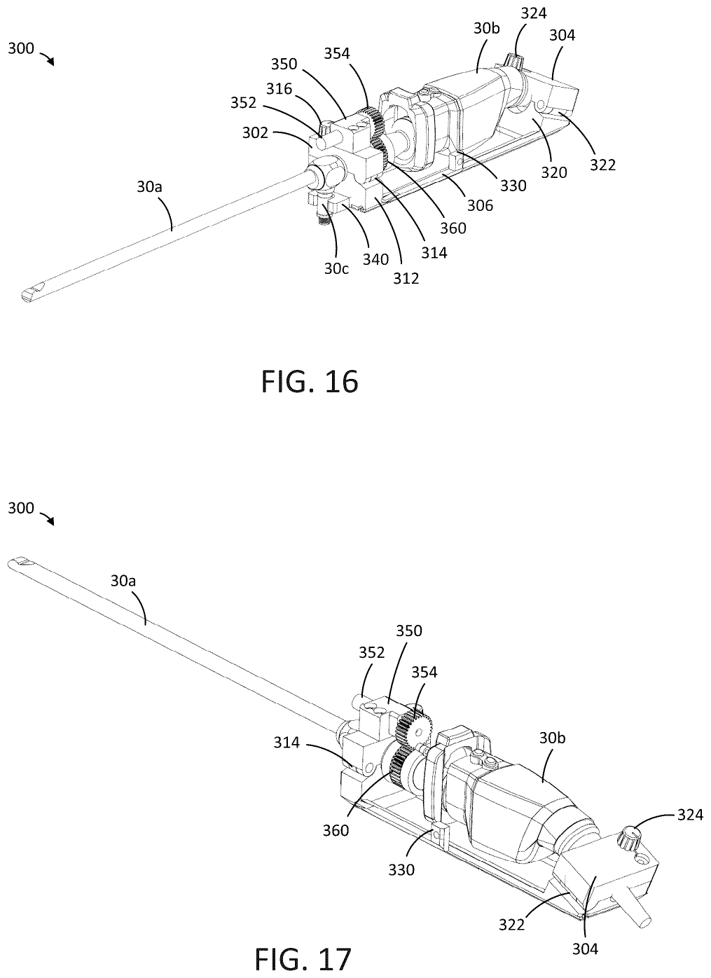

It is anticipated that the sizes of the front aperture 318 and the rear aperture 326 may be adjusted as needed to provide a firm and substantially fixed position relative to the scopes 10, 20 30. In addition, the angle between the central axis 315 and the axis 325 provide a certain degree of mechanical capture that will restrict sliding motion along axes 315, 325. However, the scopes 10, 20, 30 may also be attached to the support plate assembly 300 at additional points. Further, it is anticipated that additional points may be used to angularly orient the scopes relative to the central axis 315 and may be used to establish a consistent translation position as the support plate assembly 300 is slid into position within the grooves 128 of the scope adapter. Such additional points of connection on the support plate assembly 300 comprise tabs 330 having pinholes 332 therethrough and a post arm 340 having a post aperture 342 therethrough along an axis 345. As previously described, the tabs 330 and pinholes 332 may operate with the locking pins 250 and the extensions 130 in the same manner described with reference to the tabs 230, the pinholes 232, and FIGS. 12 and 13. Additionally, the post arm 340 and the post aperture 342 may operate with a light post 30c of the articulated scope 30 (as shown in FIG. 16) in the same manner previously described with reference to the post arm 240, the post aperture 242, and FIGS. 12 and 13.

Referring again to FIGS. 14 and 15, to support the ability of the scope adapter 100 to selectably control the adjustable angle 37 for the articulated scope 30 (as shown in FIG. 3), the support plate assembly 300 further comprises an articulation block 350, an articulation motor 352, and an articulation gear 354. The articulation block 350 couples to the front cap 302 and rotatably mounts the articulation gear 354 to the articulation motor 352 along an axis 355 via a drive shaft (not shown) through the articulation block 350. The axis 355 is substantially parallel to the central axis 315 so that the articulation gear 354 will mesh with a scope gear 360 as shown in FIGS. 16 and 17. Alternatively, other types and orientations of gears may be substituted for one or both of the articulation gear 354 and the scope gear 360, thus the axis 355 may need to be oriented differently in other embodiments. For example, the axis 355 would be orthogonal to the central axis 315 if a worm gear were substituted for one of the articulation gear 354 or the scope gear 360. In each case, the operation of the articulation motor 352 is configured to apply torque to and rotate the articulation gear 354 that then transfers torque via the scope gear 360 to rotate a portion of the articulated scope 30 that adjusts the FOV 39 via the adjustable angle 37.

Referring to FIGS. 16 and 17, the support plate assembly 300 is shown in two different isometric views and includes the articulated scope 30 installed therewith. As shown, the support plate assembly 300 is configurable to fixably couple to the scope 30 by using the front base 312 to clamp the first end 30a and by using the rear base 320 to clamp to the second end 30b. In addition, alight post 30c of the scope 30 is also captured by the post aperture 342 of the post arm 340 and thus further aligns the angular position of the scope 30 relative to the central axis 315. As best shown in FIG. 17, the scope gear 360 is shown fixably coupled to the portion of the scope 30 responsible for adjusting the FOV via the adjustable angle 37. The scope gear 360 may be slid onto the scope 30 via the first end 30a or may be otherwise clamped into position (e.g., via a two piece, hinged, or clam shell design not shown).

By any of the described methods, the support plate assembly 300 may be removably coupled to the scopes 10, 20, 30 and may be removably coupled to the inner cylinder 102. Thus by the operation of the rotational motor 110, the scopes 10, 20, 30 may be rotated with the inner cylinder 102 relative to the outer cylinder 104 to adjust the FOV 19, 29, 39 as needed for a particular surgical procedure. In addition, the operation of the articulation motor 352 may rotate the scope gear 360 and thereby adjust the FOV 39 via the adjustable angle 37.

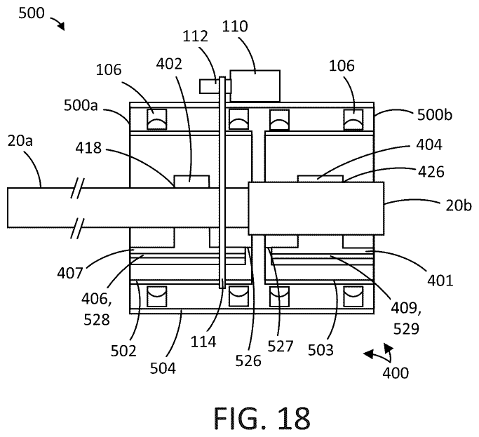

Referring to FIG. 18, an alternative embodiment of the scope adapter 100 is shown as a scope adapter 500. The configuration of the scope adapter 500 may be used with either the support plate assembly 200 or the support plate assembly 300 and may support all of the scopes 10, 20, 30 as previously described. In addition, the scope adapter 500 is similar to the scope adapter 100 in various respects and as such similar reference numerals are used to identify similar features and the explanation of such features will not be repeated in the interest of brevity except as needed for clarity. Instead, the description will focus primarily on aspects of the scope adapter 500 that are different from the scope adapter 100. Generally speaking, the scope adapter 500 supports the scope 10, 20, 30 using a pair of independent supports (e.g., a front support plate 407 and a rear support plate 401) that each rest on an independently rotatable inner cylinder.

In particular, the scope adapter 500 comprises an inner cylinder 502 and a separate rear inner cylinder 503, each within and concentrically aligned with an outer cylinder 504. The inner cylinder 502 is supported within the outer cylinder 504 by a pair of spherical bearings 106 such that the inner cylinder 502 is able to rotate around a central axis 505 while the outer cylinder 504 is fixed and is connected to the robotic arm 52 via the base 108 (shown in FIG. 5). Similarly, the rear inner cylinder 503 is supported by a pair of spherical bearings 106 such that the rear inner cylinder 503 is able to rotate around the central axis 505 independently from the rotations of the inner cylinder 502. The spherical bearings 106 may be held in position by a press fit, bonding, a series of stepped diameters within one or more of the cylinders 502, 503, 504 and may be used in combination with retention rings (e.g., “snap rings”) not shown.

The rotational motor 110 mounts in a fixed position on the outer cylinder 504. The rotational motor 110 may include a reduction gearbox (not shown) that may be used to increase or decrease the rotational speed of the shaft 112 extending therefrom. Optionally, an encoder (not shown) coupled to the rotational motor 110 or the shaft 112 may also be used to provide angular speed and/or angular position feedback. The belt 114 is operatively coupled around the shaft 112 and is operatively coupled to an outer diameter of the inner cylinder 502 by circumferentially wrapping around a portion of the inner cylinder 152. A thru hole (not shown) in the wall of the outer cylinder 504 allows passage of the belt 114.

Additionally, the inner cylinder 502 comprises a pair of shelves 526 and a pair of grooves 528, each similar to the shelves 126 and grooves 128 previously described. Similarly, the rear inner cylinder 503 also comprises a pair of rear shelves 527 and a pair of rear grooves 529, also each similar to the shelves 126 and grooves 128 previously described.

Referring still to FIG. 18 the scope adapter 500 further comprises a support plate assembly 400 that is removably coupled to the inner cylinder 502, to the rear inner cylinder 503, and to any of the surgical scopes 10, 20, 30 previously described. More specifically, the support plate assembly 400 comprises a front base 412 coupled to a front cap 402 to form a front aperture 418 that is used to fixably couple to the first end 20a of the scope 20. Optionally, the front aperture 418 may alternatively by coupled to a particular portion of the first end 20a such as the controls for selectably adjusting the adjustable angle 37 for the articulated scope 30. In this manner, rather than impart a rotation to the scope 10, 20, 30, the rotation motor 110 may be adapted to instead control the adjustable angle 37 or other features of the scopes 10, 20, 30. In addition, the support plate assembly 400 further comprises a rear base 420 coupled to a rear cap 404 to form a rear aperture 426 that is used to fixably couple to the second end 20b of the scope 20. The front support plate 407 detachably couples with the inner cylinder 502 via a slidable coupling between the groove 528 and the ridge 406. Similarly, the rear support plate 401 detachably couples with the rear inner cylinder 503 via a slidable coupling between the rear groove 529 and the rear ridge 409.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A scope adapter for adapting a surgical scope to a robotic manipulator system, the scope adapter comprising:
a rotational motor mounted in a fixed position relative to an outer cylinder;
an inner cylinder rotatably coupled to the outer cylinder; and
a support plate assembly that is removably coupled to the surgical scope and the inner cylinder.

2. The scope adapter according to claim 1, wherein the inner cylinder is rotatable concentrically inside the outer cylinder.

3. The scope adapter according to claim 1, further comprising a belt operatively coupled to the rotational motor, wherein the belt circumferentially wraps around a portion of an outer diameter of the inner cylinder, and wherein the belt is configured to transfer rotation from the rotational motor to the inner cylinder.

4. The scope adapter according to claim 1, further comprising a shelf extending from an inner diameter of the inner cylinder, where the shelf comprises a groove that is slidably couplable with a ridge extending from the support plate.

5. The scope adapter according to claim 1, further comprising a plurality of spherical bearings located between the inner cylinder and the outer cylinder.

6. The scope adapter according to claim 1, further comprising:
a scope gear fixably coupled to the surgical scope; and
an articulation motor rotatably coupled to an articulation gear, wherein the articulation gear is operably coupled to the scope gear and is configured to transfer rotations of the articulation motor to the scope gear, and thereby change an adjustable direction of a field of view for the surgical scope.

7. The scope adapter according to claim 1, wherein the support plate assembly further comprises:
a front base with a front aperture passing therethrough along a central axis of the inner cylinder; and
a rear base with a rear aperture passing therethrough, wherein the front aperture and the rear aperture are configured to fixably couple to the surgical scope.

8. The scope adapter according to claim 7, wherein the front aperture is at least partially cylindrical, and wherein the rear aperture is positioned along an axis that is non-parallel with respect to the central axis of the inner cylinder.

9. The scope adapter according to claim 7, wherein:
the front base rotatably couples to a front cap with a front hinge;
the rear base rotatably couples to a rear cap with a rear hinge; and
wherein the front aperture opens by operation of the front hinge, and the rear aperture opens by operation of the rear hinge.

10. A method of adapting a surgical scope to a robotic manipulator system with a scope adapter, the method comprising:
coupling the surgical scope to a support plate assembly;
coupling the support plate assembly to an inner cylinder;
operating a rotational motor to rotate the inner cylinder concentrically within an outer cylinder; and
rotating a portion of the surgical scope relative to the outer cylinder.

11. The method of claim 10, further comprising rotating a belt circumferentially around an outer diameter of the inner cylinder to transfer rotation from the rotational motor to the inner cylinder.

12. The method of claim 10, further comprising:
fixably coupling a scope gear to the surgical scope; and
operating an articulation motor coupled to an articulation gear to transfer rotations of the articulation motor to the scope gear, and thereby changing an adjustable direction of a field of view for the surgical scope.

13. The method of claim 10, further comprising:
coupling the surgical scope to a front base with a front aperture passing therethrough along a central axis of the inner cylinder; and
coupling the surgical scope to a rear base with a rear aperture passing therethrough.

14. The method of claim 13, further comprising:
opening the front aperture by rotating a front cap with a front hinge on the front base; and
opening the rear aperture by rotating a rear cap with a rear hinge on the rear base.

15. A scope adapter for adapting a surgical scope to a robotic manipulator system, the scope adapter comprising:
a rotational motor mounted in a fixed position relative to an outer cylinder;
an inner cylinder rotatably coupled to the outer cylinder;
a rear inner cylinder concentrically aligned with the inner cylinder;
a front support plate that is removably coupled to the surgical scope and the inner cylinder; and
a rear support plate that is removably coupled to the surgical scope and the rear inner cylinder.

16. The scope adapter according to claim 15, wherein the inner cylinder is rotatable concentrically inside the outer cylinder and relative to a rational position of the rear inner cylinder.

17. The scope adapter according to claim 15, further comprising a belt operatively coupled to the rotational motor, wherein the belt circumferentially wraps around a portion of an outer diameter of the inner cylinder, and wherein the belt is configured to transfer rotation from the rotational motor to the inner cylinder.

18. The scope adapter according to claim 15, further comprising:
a shelf extending from a first inner diameter of the inner cylinder, where the shelf comprises a groove that is slidably couplable with the front support plate; and
a rear shelf extending from a second inner diameter of the rear inner cylinder, where the rear shelf comprises a rear groove that is slidably couplable with the rear support plate.

19. The scope adapter according to claim 15, wherein:
the front support plate further comprises a front base with a front aperture passing therethrough along a central axis of the inner cylinder; and
the rear support plate further comprises a rear base with a rear aperture passing therethrough, wherein the front aperture and the rear aperture are configured to fixably couple to the surgical scope.

20. The scope adapter according to claim 19, wherein the rear aperture is positioned along an axis that is parallel with respect to the central axis of the inner cylinder.
